(19) United States
(12) Patent Application Publication
(10) Pub. No.: US 2009/0240160 A1
(43) Pub. Date: Sep. 24, 2009

(54) Infant monitoring system

(76) Inventors: Loren M. Thompson, Lapeer, MI (US); Paul T. Kotnik, Commerce Township, MI (US)

Correspondence Address: DELPHI TECHNOLOGIES, INC., M/C 480-410-202, PO BOX 5052, TROY, MI, 48007, US

(21) Appl. No.: 12/077,465

(22) Filed: Mar. 19, 2008

(51) Int. Cl. A61B 5/024; A61B 5/08

(52) U.S. Cl. 600/527; 600/534

(57) ABSTRACT

An infant monitoring system according to embodiment(s) of the present disclosure includes a positioning member having a top surface, side surface, and tension strip, and an accelerometer in operative communication with the positioning member. The top surface, which may operatively contact an infant, receives and transmits infant cardiac impulses exerted on the top surface. The side surface, operatively connected to the accelerometer, has an initial angular orientation with respect to the top surface. The side surface receives the transmitted cardiac impulses and resolves a heart rate therefrom. The strip, having a substantially low bending stiffness and substantially high tension stiffness, is operatively connected to the top surface and bends longitudinally in response to infant inhalation forces exerted on the top surface. The bending causes angular deflections of the side surface with respect to the initial angular orientation. The accelerometer detects the angular deflections and resolves therefrom a respiration rate.

### BACKGROUND

The present disclosure relates generally to monitoring, and more particularly to a system and method for monitoring infant vital sign(s).

Monitoring systems often detect movement of a person, such as an infant. As an example, it may be desirable for a caregiver to monitor a baby's movement for various reasons while the child sleeps. The systems may alert the caregiver if the child has not moved for some predetermined time. Such systems may utilize a piezoelectric crystal transducer to detect such random movements.

### SUMMARY

An infant monitoring system according to embodiment(s) of the present disclosure include an accelerometer and an infant positioning member in operative communication with the accelerometer. The positioning member includes a positioning member top surface, a positioning member side surface, and a tension strip.

The positioning member top surface is configured for operative contact with an infant having a respiration rate and a heart beat occurring at a heart rate. The top surface receives and transmits, at least in the x-axis, a plurality of cardiac impulses exerted on the top surface responsive to the heart beat.

The positioning member side surface has an initial angular orientation with respect to the top surface. The accelerometer is operatively connected to the side surface and receives therefrom the impulses transmitted along the x-axis. The accelerometer may resolve the heart rate from the received impulses.

The tension strip, which is operatively connected to the positioning member top surface, has a substantially low bending stiffness and a substantially high tension stiffness. The strip bends longitudinally in response to periodic inhalation forces exerted on the top surface due to inhalations by the infant. The periodic longitudinal bending causes respective angular deflections of the positioning member side surface with respect to the initial angular orientation.

The accelerometer detects the respective angular deflections and resolves therefrom the respiration rate.

### BRIEF DESCRIPTION OF THE DRAWINGS

Features and advantages of embodiments of the present disclosure will become apparent by reference to the following detailed description and drawings, in which like reference numerals correspond to similar, though perhaps not identical components. For the sake of brevity, reference numerals or features having a previously described function may or may not be described in connection with other drawings in which they appear.

FIG. 1 is a semi-schematic perspective view depicting an embodiment of an infant monitoring system;

FIG. 2A is a schematic front view of the embodiment of the infant monitoring system of FIG. 1;

FIG. 2B is an enlarged, cutaway, schematic front view of a portion of the embodiment of FIG. 2A;

FIG. 3 is a cutaway perspective view of another embodiment of an infant monitoring system; and

FIG. 4 is a flow diagram depicting an embodiment of a method for monitoring an infant.

### DETAILED DESCRIPTION

Monitoring systems that detect random movement may be sensitive to any moving body, including other humans or animals in addition to the infant who is being monitored. Due to the fact that the system is looking for random movement, it may be difficult to know (with a sufficient degree of certainty) that the detected movement was actually caused by the infant.

Embodiment(s) of the infant monitoring system disclosed herein advantageously monitor a heart rate as well as a respiration rate of an infant in operative communication therewith. An accelerometer, which is connected to an infant positioning member, monitors infant bodily movements due to his/her heart beat and respiration and resolves the heart rate and the respiration rate therefrom. As such, embodiment(s) of the present infant monitoring system advantageously monitor infant circulation and respiration with a single accelerometer. Such a system looks for repetitive patterns of vital signs (heart and respiration rates) that are specific to the infant being monitored. It is believed that monitoring such specific patterns reduces the occurrence of false alarms often associated with other monitoring systems.

Furthermore, embodiments of the infant monitoring system disclosed herein may advantageously be portable. The positioning member (discussed further hereinbelow) may be placed on top of any infant sleeping surface, and may be easily removed by the care giver when the infant is to be relocated. Such a portable monitor may be used in a moving vehicle. Since the system is looking for specific infant vital signs, random noise from a moving vehicle may be filtered out relatively easily.

It is to be understood that the terms “top,” “bottom,” “side,” “front,” “x-axis,” “y-axis,” “z-axis” and/or like terms are not intended to be limited to, nor necessarily meant to convey a spatial orientation, but rather are used for illustrative purposes to differentiate views of the infant monitoring system, etc. It is to be further understood that embodiment(s) of the present disclosure may be assembled/used in any suitable and/or desirable spatial orientation.

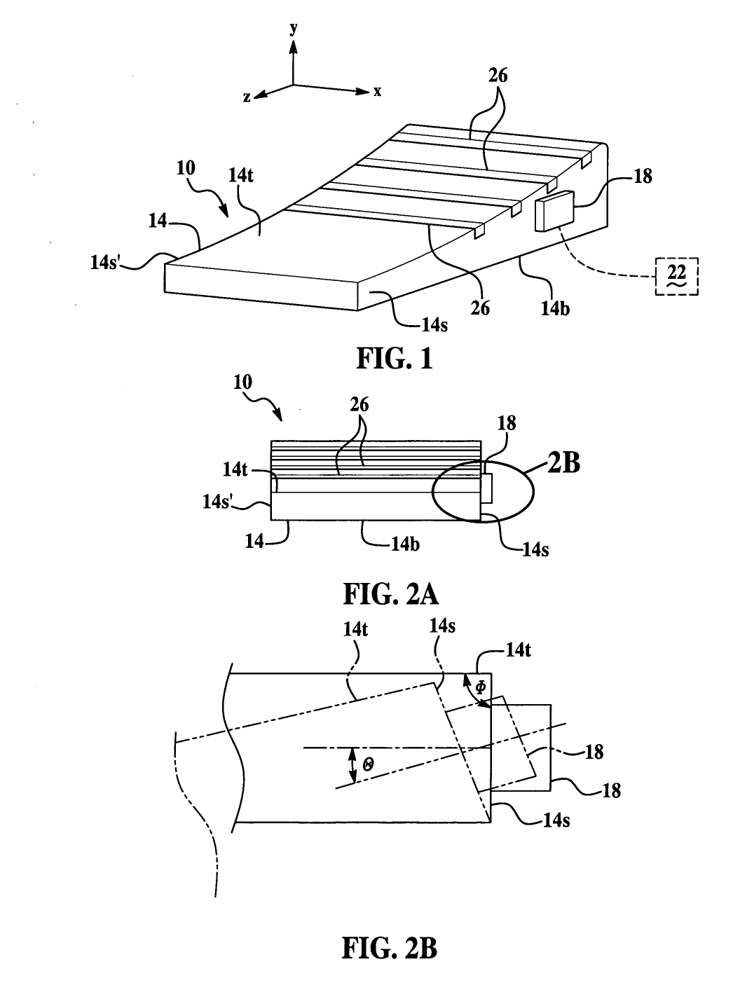

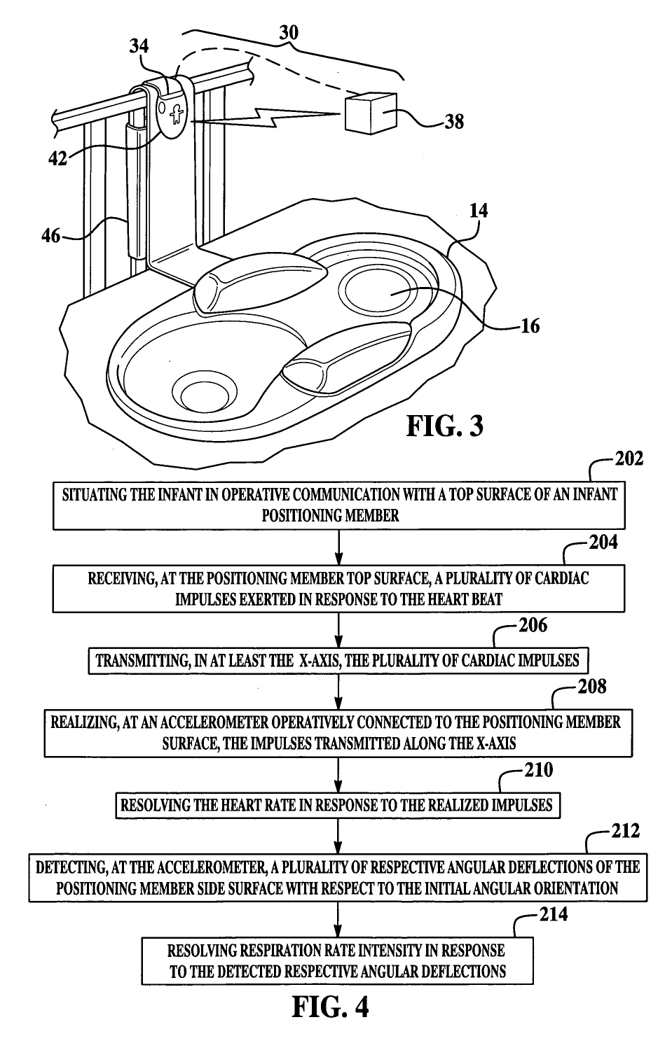

Referring now to FIG. 1, the infant monitoring system 10 includes a positioning member 14 having a positioning member top surface 14t and a positioning member side surface 14s. The positioning member may be at least partially formed from open/closed cell foam or elastomeric materials. In an embodiment, at least a portion of the positioning member 14 is substantially protected by a positioning member cover 16, as shown in FIG. 3. The cover 16 may substantially protect a portion of the positioning member 14, or the entire positioning member 14, from, for example, liquid and/or soiling. As non-limiting examples, the cover 16 may be formed at least partially from rubber and/or fabric. The cover 16 may be removable, semi-removable, or non-removable with respect to the positioning member 14.

The positioning member 14 may have any suitable geometric shape. As non-limiting examples, the positioning member 14 may be substantially round or rectangular. In an embodiment where the positioning member 14 is substantially round or oval, the positioning member 14 has a single side surface 14s. In an embodiment where the positioning member 14 is substantially square or rectangular, the positioning member includes a second positioning member side surface 14s′. In other embodiments, it is to be understood that the positioning member may have more than two side surfaces 14s. Referring also to FIG. 2B, the side surface 14s has an initial angular orientation φ with respect to the top surface 14t. The initial angular orientation φ may be defined as the angle formed between the top surface 14t and the side surface 14s as viewed along the z-axis. In the embodiment depicted in FIGS. 1, 2A and 2B, the side surface 14s meets the top surface 14t substantially at a right angle and, thus, the initial angular orientation φ is approximately 90 degrees.

The positioning member top surface 14t is configured for operative contact with an infant. It is to be understood that clothing, bedding, and/or the like may be disposed between the infant and the top surface 14t during use/operation of the infant monitoring system 10. Placing the infant substantially near the center of the positioning member 14 (i.e., approximately halfway between the side surfaces 14s, 14s′) may be desirable to prevent the infant from shifting off of the positioning member 14, which shifting may prevent the infant monitoring system 10 from properly functioning. Top surface 14t may have any shape suitable for operative contact with the infant. As non-limiting examples, the top surface 14t may be substantially flat (either horizontal or tilted) or substantially concave. In an embodiment, the top surface 14t is configured to substantially maintain the position of the infant on the positioning member 14 while the infant is in operative communication therewith. As an example, the top surface 14t depicted in FIG. 3 is contoured to substantially mirror an infant's shape.

Referring back to FIG. 1, cardiac impulses may be exerted on the top surface 14t by the infant in response to the infant's heart beat. The top surface 14t receives and transmits, at least in the x-axis, the cardiac impulses. As such, the cardiac impulses are transmitted, by the top surface 14t, substantially toward the positioning member side surface 14s. Generally, the cardiac impulses are communicated to an accelerometer 18 as a mechanical vibration/impulse. The frequency of the signal is within a range to which the accelerometers 18 is very sensitive, and, as such, may be easily resolved.

The accelerometer 18 is in operative communication with the positioning member side surface 14s. In an embodiment, the accelerometer 18 is not a piezoelectric device. Non-limiting examples of suitable accelerometers 18 include silicone capacitive accelerometers or microelectromechanical system (MEMS) inclinometers. The accelerometer 18 receives the cardiac impulses transmitted along the x-axis from the positioning member top surface 14t. In an embodiment, the accelerometer 18 resolves the infant's heart rate based upon the received cardiac impulses. In another embodiment, the accelerometer 18 is in operative communication with a component 22, which receives a signal indicative of the cardiac impulses from the accelerometer 18 and resolves the heart rate therefrom. The accelerometer may be substantially insensitive to substantially low-frequency (e.g., ranging from about 0.2 to about 0.8 hertz) movement in the z-axis resulting from infant inhalations.

In an embodiment, the heart rate is resolved by monitoring the number of cardiac impulses received by the accelerometer 18 during a predetermined length of time. The number of received cardiac impulses may then be divided by the predetermined length of time to calculate the heart rate.

The positioning member top surface 14t is operatively connected to one or more tension strips 26, each having a substantially low bending stiffness and a substantially high tension stiffness. In an embodiment, the tension strip 26 is formed from an adhesive backed thin polyester film. It is to be understood that the tension strip 26 may be formed from any thin material which provides sufficient lateral stiffness and minimal bending stiffness. Generally, the tension strip 26 provides bending stiffness that is comfortable for the user, and has sufficient lateral stiffness to provide ample rotational movement which an accelerometer 18 is able to resolve. Such lateral and bending stiffness may be achieved by skinning an open/closed cell material, such as a two density urethane foam product. In an embodiment having a plurality of tension strips 26, the tension strips 26 are disposed substantially parallel with each other. The tension strip 26 may be connected to the top surface 14t or may be embedded within the body of the positioning member 14 near the top surface 14t or such that the tension strip 26 acts on the top surface 14t, as will be discussed further below. The tension strip 26 may extend along the top surface 14t substantially to the side surface 14s or may, alternately, extend to the side surface 14s and, further, a portion of the distance along the side surface 14s (i.e., along the y-axis). The tension strip 26 responds to inhalation forces exerted on the top surface 14t, which are responsive to infant inhalations, by bending longitudinally (i.e., substantially about the z-axis).

Referring now to FIGS. 1, 2A and 2B together, the longitudinal bending of the tension strip 26 causes an angular deflection Θ of the positioning member top surface 14t with respect to the initial angular orientation φ, which angular deflection Θ is shown in phantom in FIG. 2B. The accelerometer 18 detects the angular deflection Θ and resolves the respiration rate therefrom. In an embodiment, the component 22 receives a signal from the accelerometer 18 indicative of the angular deflection η and resolves the respiration rate therefrom. It is to be understood that a single accelerometer (and not more than one accelerometer) may both receive the cardiac impulses and detect the angular deflections Θ. As such, the single accelerometer 18 may resolve the heart rate and the respiration rate.

In an embodiment, each angular deflection Θ that is larger than a predetermined angle is associated with an infant inhalation. Generally, the angular deflection Θ is large enough to be resolved by the accelerometer 18. The repeating nature of the signal and its frequency/frequency stability indicate that the deflection is an inhalation signal. The respiration rate may be resolved by monitoring the number of inhalations detected by the accelerometer 18 during a predetermined length of time. The number of received inhalations may then be divided by the predetermined length of time to calculate the respiration rate.

It is to be understood that longitudinal bending occurs when an inhalation force, caused by an infant's expanding lungs and, thus, abdomen, press on a portion of the tension strip 26, substantially in the y-axis. As such, the inhalation force presses on a portion of the top surface 14t (i.e., substantially along the y-axis), which causes the longitudinal bending of the tension strip 26 having the substantially low bending stiffness. Further, since the tension strip 26 has a substantially high tension stiffness, the longitudinal bending causes the tension strip 26 to “act on” or pull, in the x-axis, the top of the positioning member 14 toward the point of receipt of the inhalation force (e.g., substantially near the longitudinal center of the tension strip 26). It is to be understood that the angular deflection Θ occurs when the longitudinal bending compresses the positioning member top 14t while the bottom remains substantially non-deformed.

Referring to FIG. 3, an alert system 30 may be in operative communication with the accelerometer 18. It is to be understood that FIG. 3 depicts another embodiment of the positioning member 14.

The alert system 30 emits an alarm if the heart rate and/or the respiration rate extends beyond a respective predetermined range. In an embodiment, the alarm is emitted from a transmitter 34 in operative communication with the accelerometer 18. In another embodiment, the alert system 30 transmits a signal from the transmitter 34 to a receiver 38 via a wired or wireless connection, which signal triggers emission of the alarm from the receiver 38. It is to be understood that an alert system 30 may include two or more receivers 38 which may emit the alarm substantially simultaneously upon triggering. The transmitter 34 and the receiver 38 may be powered by a power cord, a replaceable power source, such as one or more batteries, and/or a rechargeable power source.

The alarm may include an audible alarm, a visual alarm, and/or a tactile alarm. In an embodiment, the alarm is audibly output (played, provided, etc.) via speakers in operative communication with the transmitter 34 and/or the receiver 38. In a non-limiting example, the audible alarm includes a verbal message and/or one or more sounds, such as beeps. In another embodiment, the transmitter 34 and/or the receiver 38 includes a notification panel which digitally displays a visual notice to the user. In a non-limiting example, the visual notice may include a textual message and/or a blinking light. In yet another embodiment, the alarm is a tactile alarm, which vibrates the receiver 38, which may be embodied, for example, as a wristband, a clip (e.g., configured for attachment to personal garments, bedding, etc.), and/or the like. It is to be understood that the alarm may be presented on divergent media, such as, for example, an alarm that is both visual and audible, and is presented to a user substantially simultaneously.

As an example, the alert system may emit the alarm if the respiration rate extends beyond the predetermined range, which predetermined range may be from about 12 breaths per minute to about 60 breaths per minute. As another non-limiting example, the predetermined range for the respiration rate may be from about 30 breaths per minute to about 60 breaths per minute. In still another example, the alert system may emit the alarm if the heart rate extends beyond the predetermined range, which predetermined range may be from about 60 beats per minute to about 150 beats per minute. As another non-limiting example, the predetermined range for the heart rate may be from about 100 beats per minute to about 120 beats per minute.

It is to be understood that the predetermined range may vary from person to person, and may depend, at least in part, on the age of the person and/or the shape in which the person is in. For an infant ranging in age from zero to six months, the predetermined respiration rate range may be from about 30 breaths per minute to about 50 breaths per minute and the predetermined heart rate range may be from about 120 beats per minute to about 140 beats per minute. For an infant ranging in range from about six months to about twelve months, the predetermined respiration rate range may be from about 25 breaths per minute to about 40 breaths per minute and the predetermined heart rate range may be from about 95 beats per minute to about 120 beats per minute. Generally, the older the infant is, the lower the respiration and heart rates are. Furthermore, such ranges may be increased or decreased if the infant, for example, suffers from a heart and/or respiratory condition.

If it is desirable to monitor an adult's vital signs using the monitoring system 10, the predetermined ranges may be less than those of an infant. For example, the predetermined respiration rate range for an adult may be from about 15 breaths per minutes to about 20 breaths per minute, and the predetermined heart rate range for an adult may be from about 70 beats per minutes to about 85 beats per minute.

The transmitter 34 may be operatively connected to the positioning member 14 in a removable manner. As a non-limiting example, the transmitter 34 may be situated in a pocket 42 formed in the positioning member 14. In the embodiment depicted in FIG. 3, the transmitter 34 is situated in a pocket 42 formed in an attachment member 46. The attachment member 46 may releasably connect the positioning member 14 to a substantially stationary apparatus, such as a crib. In an embodiment, the attachment member 46 utilizes one or more of a snap, button, hook-and-eye, hook-and-loop, and/or the like to attach the positioning member 14 to the substantially stationary apparatus. The attachment member 46 may be formed from flexible and/or rigid materials.

In an embodiment, the infant monitoring system 10 includes components to operate as an audio monitor. As such, the transmitter 34 may include a speaker to pick up noises, which noises (or a signal indicative thereof) may be transmitted to the receiver 38 and output therefrom audibly, visually, and/or tactilely.

Referring to FIG. 4, an embodiment of a method of monitoring an infant includes situating the infant in operative communication with the top surface 14t of an infant positioning member 14, as depicted at reference numeral 202; receiving, at the top surface 14t, a plurality of cardiac impulses exerted in response to the infant's heart beat, as depicted at reference numeral 204; and transmitting, in the x-axis, the plurality of cardiac impulses, as depicted at reference numeral 206. The accelerometer 18 realizes the transmitted impulses, as depicted at reference numeral 208; and the heart rate is resolved in response to the realized impulses, as depicted at reference numeral 210. The embodiment further includes detecting, at the accelerometer 18, a plurality of respective angular deflections Θ of the side surface 14s with respect to the initial angular orientation φ, as depicted at reference numeral 212; and resolving the respiration rate in response to the detected respective angular deflections Θ, as depicted at reference numeral 214.

It is to be understood that the terms “connect/connected/connection” and/or the like are broadly defined herein to encompass a variety of divergent connected arrangements and assembly techniques. These arrangements and techniques include, but are not limited to (1) the direct communication between one component and another component with no intervening components therebetween; and (2) the communication of one component and another component with one or more components therebetween, provided that the one component being “connected to” the other component is somehow in operative communication with the other component (notwithstanding the presence of one or more additional components therebetween). Additionally, two components may be permanently, semi-permanently, or releasably engaged with and/or connected to one another.

While several embodiments have been described in detail, it will be apparent to those skilled in the art that the disclosed embodiments may be modified. Therefore, the foregoing description is to be considered exemplary rather than limiting.

What is claimed is:

1. An infant monitoring system, comprising:
an accelerometer; and
an infant positioning member in operative communication with the accelerometer, the positioning member including:
a positioning member top surface configured for operative contact with an infant having a respiration rate and a heart beat occurring at a heart rate, the top surface configured to receive and transmit, at least in the x-axis, a plurality of cardiac impulses exerted on the top surface responsive to the heart beat;
a positioning member side surface having an initial angular orientation with respect to the top surface, the side surface having the accelerometer operatively connected thereto and configured to: receive from the side surface the impulses transmitted along the x-axis; and resolve therefrom the heart rate; and
a tension strip operatively connected to the positioning member top surface, the tension strip having a substantially low bending stiffness and a substantially high tension stiffness, the strip configured to bend longitudinally, in response to periodic inhalation forces exerted on the top surface in response to inhalations by the infant, the periodic longitudinal bending configured to cause respective angular deflections of the positioning member side surface with respect to the initial angular orientation, the accelerometer further being configured to: detect the respective angular deflections; and resolve therefrom the respiration rate.

2. The infant monitoring system of claim 1 wherein the accelerometer is not more than a single accelerometer configured to both receive the plurality of the cardiac impulses and detect the respective angular deflections.

3. The infant monitoring system of claim 2 wherein the accelerometer is substantially insensitive to movement in the z-axis having a frequency ranging from about 0.2 hertz (Hz) to about 0.8 hertz (Hz).

4. The infant monitoring system of claim 1 wherein the tension strip is a material which provides a predetermined lateral stiffness and a predetermined bending stiffness.

5. The infant monitoring system of claim 1, further comprising a positioning member cover configured to at least partially protect the infant positioning member, the positioning member cover being removable, semi-removable, or non-removable.

6. The infant monitoring system of claim 1, further comprising an alert system in operative communication with the accelerometer and configured to emit an alarm if at least one of the respiration rate or the heart rate extends beyond a respective predetermined range.

7. The infant monitoring system of claim 6 wherein the alert system is configured to emit the alarm if the respiration rate extends beyond the respective predetermined range, and wherein the respective predetermined range extends from about 12 breaths per minute to about 60 breaths per minute.

8. The infant monitoring system of claim 6 wherein the alert system is configured to emit the alarm if the heart rate extends beyond the respective predetermined range, and wherein the respective predetermined range extends from about 60 (beats per minute) to about 150 (beats per minute).

9. The infant monitoring system of claim 6 wherein the alarm includes at least one of an audible alarm, a visual alarm, or a tactile alarm.

10. The infant monitoring system of claim 1 wherein the infant positioning member is at least partially formed from at least one of an open/closed cell foam or an elastomeric material.

11. A method of monitoring an infant having a respiration rate and a heart beat occurring at a heart rate, the method comprising:
situating the infant in operative communication with a top surface of an infant positioning member, the positioning member also having a positioning member side surface having an initial angular orientation with respect to the top surface, the top surface operatively connected to a tension strip having a substantially low bending stiffness and a substantially high tension stiffness;
receiving, at the positioning member top surface, a plurality of cardiac impulses exerted in response to the heart beat;
transmitting, at least in the x-axis, the plurality of cardiac impulses;
realizing, at an accelerometer operatively connected to the positioning member side surface, the impulses transmitted along the x-axis;
resolving the heart rate in response to the realized impulses;
detecting, at the accelerometer, a plurality of respective angular deflections of the positioning member side surface with respect to the initial angular orientation, the respective angular deflections responsive to periodic longitudinal bending of the tension strip caused by periodic inhalation forces exerted on the top surface in response to inhalations by the infant; and
resolving the respiration rate in response to the detected respective angular deflections.

12. The method of claim 11 wherein realizing the transmitted impulses and detecting the responsive angular deflections is performed by not more than one accelerometer.

13. The method of claim 11, further comprising emitting an alarm from an alert system if at least one of the respiration rate or the heart rate extends beyond a respective predetermined range.

14. The method of claim 13 wherein the alert system emits the alarm if the respiration rate extends beyond the respective predetermined range, and wherein the respective predetermined range extends from about 12 breaths per minute to about 60 breaths per minute.

15. The method of claim 13 wherein the alert system emits the alarm if the heart rate extends beyond the respective predetermined range, and wherein the respective predetermined range extends from about 60 (beats per minute) to about 150 (beats per minute).

16. The method of claim 13 wherein the alarm includes at least one of an audible alarm, a visual alarm, or a tactile alarm.
